(19) United States
(12) Patent Application Publication
(10) Pub. No.: US 2023/0235351 A1
(43) Pub. Date: Jul. 27, 2023

(54) GENE XA7 IN RICE CONFERS A RESISTANCE TO XANTHOMONAS ORYZAE PV. ORYZAE

(71) Applicant: Zhejiang Normal University, Jinhua (CN)

(72) Inventors: Xifeng Chen, Jinhua (CN); Bojun Ma, Jinhua (CN); Pengcheng Liu, Jinhua (CN); Le Mei, Jinhua (CN); Hui Liu, Jinhua (CN); Zhandong Ji, Jinhua (CN); Long Chen, Jinhua (CN); Xixi Zheng, Jinhua (CN); Yuchen Zhang, Jinhua (CN)

(21) Appl. No.: 18/119,854

(22) Filed: Mar. 10, 2023

Related U.S. Application Data
(63) Continuation-in-part of application No. PCT/CN2020/141383, filed on Dec. 30, 2020.

(30) Foreign Application Priority Data
Sep. 10, 2020 (CN) 202010943795.5

(51) Int. Cl. C12N 15/82; A01H 6/46

(52) U.S. Cl. CPC C12N 15/8281; A01H 6/4636

(57) ABSTRACT

The invention refers to the function and application of a disease-resistance gene Xa7 highly resistant to the bacterial blight of rice, belonging to the field of plant genetics. The invention discloses a gene Xa7 with high resistance to rice bacterial blight. The nucleotide sequence of gene Xa7 is shown in SEQ ID No: 1. The invention also provides the usage of the gene Xa7 to improve the resistance of rice to bacterial blight.

### CROSS-REFERENCE TO RELATED APPLICATIONS

The present application is a continuation-in-part application of PCT application No. PCT/CN2020/141383 filed on Dec. 30, 2020, which claims the benefit of Chinese Patent Application No. 202010943795.5 filed on Sep. 10, 2020, each of which is incorporated by reference herein in its entirety.

### REFERENCE TO SEQUENCE LISTING

The Sequence Listing XML file submitted via the USPTO Patent Center, with a file name of “Sequence_Listing_ZZZHCH-21001-USPT.xml”, a creation date of Mar. 10, 2023, and a size of 32.0 KB, is part of the specification and is incorporated in its entirety by reference herein.

### TECHNICAL FIELD

The disclosure refers to a high resistant gene Xa7 against rice bacterial-blight disease caused by Xanthomonas oryzae pv. oryzae, belonging to the field of plant genetics.

### BACKGROUNDS

Rice (Oryza sativa L.) is one of the most important food crops worldwide, and more than half of the world's population take rice as the staple food. However, rice is always damaged by various diseases in the production in field, which seriously affects its high and constant yield as well as the quality of grain, threatening the food security of China and the whole world.

Bacterial blight caused by Xanthomonas oryzae pv. oryzae (Xoo) is one of the most serious bacterial diseases of rice in production in China, and also is one of the three major diseases of rice. Before the 1980s, bacterial blight frequently broke out and led the rice yield to reduce by 20% to 30%, and up to 50% in the severe situation, or Even lost all of the harvest (Niño-Liu et al. 2006). The disease has been effectively controlled by introducing the resistant genes, such as Xa4 and Xa21, into the mainly cultivated varieties of rice in China. However, with the global warming and the continuous variation of Xoo, new kind pathogens have emerged inevitably and the mainly cultivated varieties of rice gradually lose the disease resistance to bacterial blight (Zhang. 2009). Recently, bacterial blight has been aggravated year by year, and the problem of “reemergence of the old disease” has become serious increasingly. According to the statistics, the occurrence area of bacterial blight in China is more than 10 million Mu since 2017 (Chen et al. 2019). The yield loss is huge, which is a serious threat to food security. There is an urgent need to introduce new genes resources resistance to bacterial blight. At the moment, characterization of new disease-resistance genes with broad spectrum and durability from plants and analysis of the resistant molecular mechanism of these genes have become a research hotspot of plant immunity and an important goal of crop breeding globally (Xu et al. 2019; Li et al. 2020).

Gene Xa7 is dominant, high-resistance, broad-spectrum, durable and hot-tolerance to bacterial blight of rice, and also is a “star gene” well recognized internationally as the most durable resistance gene to bacterial blight (Vera Cruz et al. 2000; White et al. 2009; Zhang et al. 2015). Therefore, this gene has a great value and application prospect in breeding. More than 40 years ago, the Xa7 gene was originally identified from a Bangladesh rice cultivar named DV85 in 1970s (Sidhu et al. 1978). Since the disease-resistance performance of this gene is very unique, many scientific research labs in different countries have focused on the clone of Xa7 gene for few decades. This gene was initially located at 107.5 cM of rice chromosome 6 by Kaji et al. (1995). Subsequently, it had been finely mapped into a 2.7-cM region by Porter et al. (2003), and then been narrowed into 118.5 kb by Chen et al. (2008). However, the Xa7 gene still was not identified, and its molecular mechanism of disease resistance was unknown. The team of inventors studied the Xa7 gene from 2006, and initially located this gene in the 200-kb region on rice chromosome 6 (Zhang et al. 2009).

### SUMMARY OF THE INVENTION

The technical problem of this invention is to clone a new gene with high resistance to bacterial-blight disease of rice and provide its usage in application.

In order to solve the technical problem above, this invention provides a gene named Xa7 with high resistance to bacterial blight of rice, the nucleotide sequence of the gene is shown as SEQ ID NO: 1, and the amino-acid sequence of the encoding protein is shown as SEQ ID NO: 2.

The invention also provides the usage of this gene: significantly increasing the bacterial-blight resistance of rice.

As an improvement of this invention, the usage of the gene Xa7 with highly resistance to rice bacterial blight is to construct the expression vector of Xa7 gene. The expression vector of Xa7 gene obtained contains a 1325-bp DNA sequence (SEQ ID No: 3), which contains the 342-bp sequence of gene Xa7 (SEQ ID NO: 2), its promoter sequence (439 bp) at the upstream of Xa7, and its terminal signal sequence (544 bp) of transcription at the downstream of Xa7.

As an improvement of this invention, the usage of the gene Xa7 with highly resistance to rice bacterial blight is to design a gene-editing target sequence as Target-1: 5′-CGTATGCCCGTTGCAGTTGCAGG-3′ (SEQ ID NO. 4), and to obtain the knockout vector of Xa7 gene with Target-1.

As an improvement of this invention, the usage of the gene Xa7 with highly resistance to rice bacterial blight is to design a gene-editing target sequence as Target-2: 5′-CCAGTTCCCGCGCGCCGCTGGGG-3′ (SEQ ID NO. 5), and to obtain the knockout vector of Xa7 gene with Target-2.

The team of inventors studied the Xa7 gene from 2006, and overcoming all difficulties during a decade, has cloned the Xa7 gene finally using various techniques.

The invention further provides a recombinant vector including a nucleotide sequence encoding the amino acid sequence of SEQ ID NO: 2.

In one embodiment, the recombinant vector includes the nucleotide sequence of SEQ ID NO: 1 or 3, preferably SEQ ID NO: 3.

The recombinant vector may be any vector that is capable of expressing the gene Xa7 in a rice plant. In one embodiment, the recombinant vector a plasmid.

The invention also provides a transgenic rice plant including the above recombinant vector.

The invention further provides a method for increasing resistance of a rice plant against the bacterial-blight disease, the method including:

transforming a recombinant vector including a nucleotide sequence encoding the amino acid sequence of SEQ ID NO: 2 into the rice plant, resulting a transgenic rice plant capable of expressing a protein having the amino acid sequence of SEQ ID NO: 2.

In one embodiment, the method may further include a step of amplifying the nucleotide sequence of SEQ ID NO: 1 through polymerase chain reaction.

In one embodiment, the method may further include a step of introducing the amplified nucleotide sequence into a gene expression vector.

In one embodiment, the amplification may be performed through a pair of primers as set forth in SEQ ID NOs: 14 and 15.

The technical proposal of the invention is as follows:

Based on the preliminary works, the map-based cloning technique was used in this invention. The bacterial-blight resistance rice variety Zhen-hui 084 (containing Xa7 gene) and the bacterial-blight susceptible rice variety Cheng-hui 448 was crossed, and the F₁ plants were self-crossed to build an F₂ population with more than 10,000 individual plants from the Xa7 gene fine mapping. After identification of bacterial-blight resistance by the inoculation of Xoo strain PXO86 (from International Rice Research Institute), more than 2,000 susceptible individual plants from the F2 population were selected for genetic analysis, and the Xa7 gene was further mapped into 50 kb (According to the genome of the sequenced rice variety Nipponbare). However, in the Xa7 mapping interval, primers designed based on the Nipponbare genome could not amplify any interest band by PCR from the rice varieties containing Xa7 genes, such as Zhen-hui 084 and IRBB7. It was speculated that there may be a large number of unknown sequences in this region. Therefore, the genomic libraries of Zhen-hui 084, IRBB7 and its susceptible Near-Isogenic Line IR24 were constructed respectively, and the clones for making the contigs which covers the Xa7 mapping region were screened from the libraries for each variety. The sequencing results showed that the actual physical distance of the Xa7 mapping region both Zhen-hui 084 and IRBB7 was 120 kb with a large number of repeats sequences, and the 120-kb sequences of the two varieties were identical. However, the physical distance of IR24 in this region was 50 kb, which is mainly the same as that of Nipponbare. However, the 120 kb of the rice varieties containing Xa7 gene had almost no homology with the 50 kb of Nipponbare and IR24, and the map-based cloning technique could not be used to narrow the mapping interval.

Besides cloning the Xa7 gene, the applicants mutagenized the seeds of Zhen-hui 084 by radiation to screen the mutation suppressors of Xa7 gene from more than 20,000 mutagenic lines, and several mutants highly susceptible to the bacterial-blight strains PXO86 were obtained. Through PCR amplification of the Xa7 mapping region in these mutants, a mutant zsm-2 was found to have a deletion of chromosome fragment, which was just overlap with the Xa7 mapping region. The sequence of the deletion fragment was determined as 106 kb through the high-throughput sequencing, which has 25 kb overlapping with the Xa7 mapping region. Therefore, the Xa7 gene was further delimited in the range of 25 kb. Based on these results, the applicants constructed four expression vectors of subclones covering the 25-kb region, and obtained stable transgenic plants of the susceptible rice variety Zhong-hua 11 by genetic transformation. The results showed that the transgenic lines containing the two subclones were highly resistant to strain PXO86, and the overlapping part contained only a new gene (SEQ ID NO: 1) with unknown function, which had no homology with the DNA of known genes and the amino-acid sequences of their encoding proteins. In order to verify this gene function, the sequence of this gene was obtained from the bacterial-blight resistance variety Zhen-hui 084 by PCR to construct into an expression vector, which was genetically transformed into the rice variety Zhong-hua 11 highly susceptible to bacterial blight. It was found that the transgenic positive plants were highly resistant to bacterial blight (FIG. 3). At the same time, the CRISPR/Cas9 technology was used to knock out this gene in the bacterial-blight resistance variety Zhen-hui 084, and the gene-knockout plants was highly susceptible to bacterial blight (FIG. 4). Therefore, this gene is the gene Xa7 with highly resistance to rice bacterial blight both by forward and reverse genetics approaches in the invention.

### BRIEF DESCRIPTION OF THE DRAWINGS

The specific embodiments of the invention are described in detail below with reference to the accompanying drawings.

FIG. 1. PCR identification of Xa7 transgenic plants of Zhong-hua 11.

In FIG. 1:

M refers to DNA marker DL5000; Zhen-hui 084 is the positive control; Zhong-hua 11 is the negative control; T-1 to T-4 plants are different positive transgenic plants; T-5 and T-6 plants are non-transgenic negative plants.

FIG. 2. The sequences of knockout targets to Xa7 gene by CRISPR/Cas9 and the mutation types of the knockout transgenic plants;

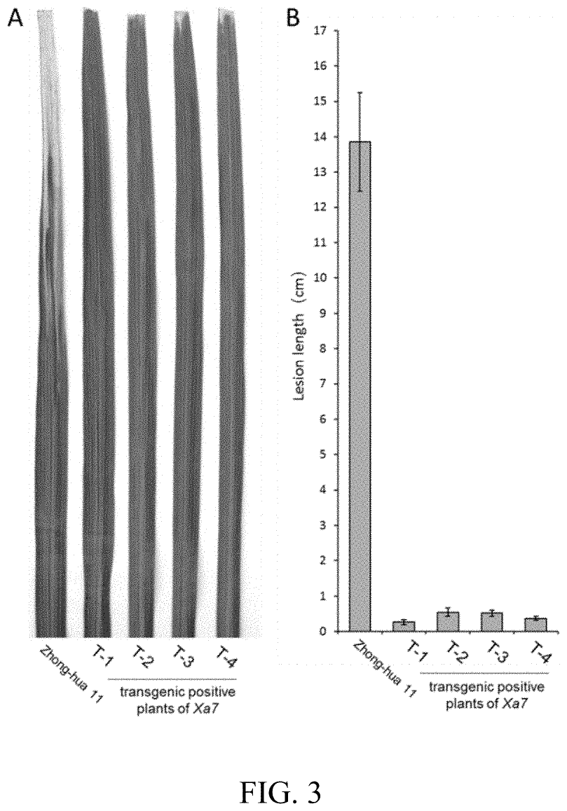

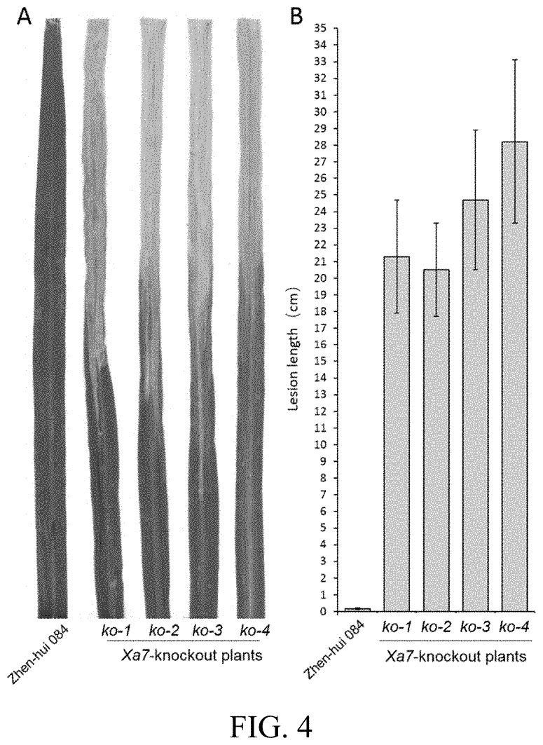

In FIG. 2:

A refers to the sequences and sites of the two target sites Target-1 (SEQ ID NO. 4) and Target-2 (SEQ ID NO. 5) to knockout the Xa7 gene by CRISPR/Cas9

B refers to the two types of Xa7 gene mutants obtained using Target-1 of knockout vector; among, WT refers to the wild-type sequence unedited; in the Xa7 gene locus, the ko-1 knockout plants contain one-base (C) insertion on a chromosome (SEQ ID NO. 6) and two-bases (TT) deletion on the other chromosome (SEQ ID NO. 7); in the Xa7 gene locus, the ko-2 knockout plants contain one-base (T) insertion on a chromosome (SEQ ID NO. 8) and 14-bases deletion on the other chromosome (SEQ ID NO. 9);

C refers to the two types of Xa7 gene mutants obtained using Target-2 of knockout vector; among, WT refers to the wild-type sequence unedited; in the Xa7 gene locus, the ko-3 knockout plants contain one-base (C) insertion on a chromosome (SEQ ID NO. 10) and 30-bases deletion on the other chromosome (SEQ ID NO. 11); in the Xa7 gene locus, the ko-4 knockout plants contain four-bases deletion on a chromosome (SEQ ID NO. 12) and one-base (T) insertion on the other chromosome (SEQ ID NO. 13).

FIG. 3. Bacterial-blight resistance identification of the Xa7-transgenic plants of Zhong-hua 11.

Zhong-hua 11 is a control variety susceptible to bacterial blight and no Xa7 gene. T-1, T-2, T-3 and T-4 are different lines of Xa7 positive transgenic plants. The infected leaves were taken pictures and measured two weeks after inoculation with Xoo strain PXO86. A refers to the pictures of lesion length of inoculated leaves; B refers to the measurement and statistics of lesion length of inoculated leaves.

FIG. 4. Bacterial-blight resistance identification of the Xa7-knockout plants of Zhen-hui 084.

Zhen-hui 084 is a bacterial-blight resistance variety with the Xa7 gene. ko-1, ko-2, ko-3 and ko-4 are different types of Xa7-knockout plants. The infected leaves were taken pictures and measured two weeks after inoculation with Xoo strain PXO86. A refers to the pictures of lesion length of inoculated leaves; B refers to the measurement and statistics of lesion length of inoculated leaves.

### DETAILED DESCRIPTION OF THE EMBODIMENTS

### Example 1. Introducing the Xa7 Gene into Zhong-Hua 11, a Bacterial-Blight Susceptible Rice Variety

### (1) PCR Amplification of the Xa7 Gene

PCR primers were designed and synthesized as the following sequences:

forward:
(SEQ ID NO. 14)
5′-cggccagtgccaagcttGTTCGGCTGCGGCCTGCAG-3′,
reverse:
(SEQ ID NO. 15)
5′-tgaccacccggggatccAATCAGTTGTGAGGCCGGTT-3′.

The sequence underlined are used for homologous recombination into the vector. The genomic DNA of rice variety Zhen-hui 084 was used as template to amplify the Xa7 gene by PrimeSTAR® HS DNA Polymerase (Takara, Japan). Total 50-μl PCR solution contained 25 μl Primestar HS (Premix), 1 μl forward and 1 μl reverse primers (10 μm), 2 μl template DNA (<200 ng) and 21 μl sterile water. PCR procedure was as follows: pre-denaturation at 95° C. for 5 min; denaturation at 98° C. for 10 sec; annealing at 58° C. for 15 sec; extension at 72° C. for 90 sec, 30 cycles; extension at 72° C. for 5 min.

The sequence of SEQ ID No: 3 was obtained including the Xa7 gene (SEQ ID NO: 1).

### (2) Construction of Xa7-Expression Vector

The pCAMBIA-1300 vector was obtained from Abcam (Shanghai, China) through commercial purchase, and the link is https://www.abcam.cn/. This vector was digested by two restriction endonuclease Hind III and BamH I (Takara, Japan). The reaction system was as follows: Hind III 1 μl, BamH I 1 μl, 4 μl buffer (coming with the products), 15 μl pCAMBIA-1300 vector plasmid, ddH₂O was added up to 40 μl, under 37° C. incubating for 4 h. The digested products were purified by the PCR Cleaning Kit (Axygen, USA) according to the manufacturer's instructions. The PCR products of Xa7 gene were purified by the same method. Then, the ligation reaction was performed by the homologous recombination Kit of TransGen company (pEASY®-Uni Seamless Cloning and Assembly Kit), and the reaction solution contains 1.25 μl vector products, 3.75 μl PCR products, 5 μl Assembly mix, incubating at 50° C. for 15 min. The reaction product was transformed into competent cells JM109 by Heat-Shock method, and the positive clones were obtained. Then, the positive clones were sent to biotechnology company for sequencing to verify the sequence of the inserted fragment (SEQ ID No: 3) in the vector, and the expression vector of Xa7 gene was obtained.

### (3) Rice Genetic Transformation and Identification of Positive Transgenic Plants

The expression vector of Xa7 gene obtained above was transformed into a bacterial-blight susceptible rice variety Zhong-hua 11 to obtain the transgenic rice plants by the method reported by Nishimura et al. (Nishimura et al, Nat Protoc, 2006).

Genomic DNA was extracted from the leaves of transgenic plants according to the method of Panaud et al. (1996). The primers were synthesized as follows: forward: 5′-GTTCGGCTGCGGCCTGCAG-3′ (SEQ ID NO. 16) and, reverse: 5′-AATCAGTTGTGAGGCCGGTT-3′ (SEQ ID NO. 17). PCR amplification was performed with 2×Taq PCR Master Mix (TianGen, China).

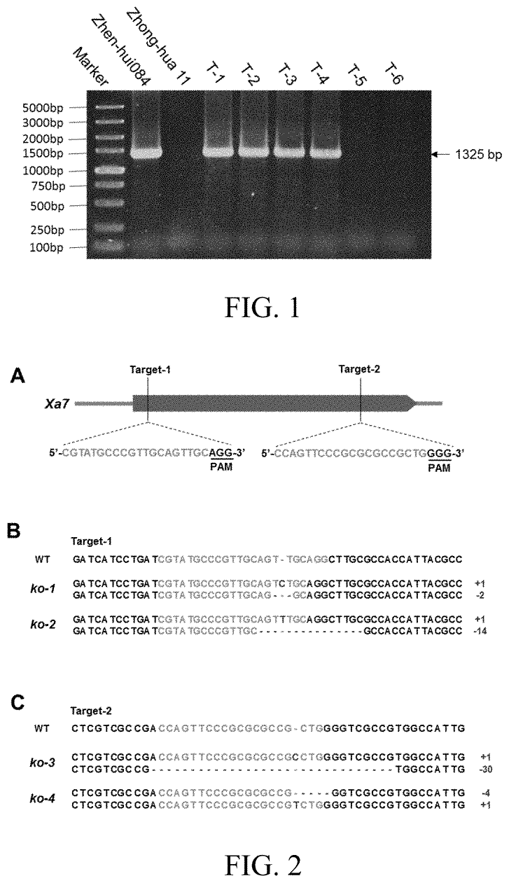

Total 20 μl PCR solution contains 2×Taq PCR Master Mix 10 μl, forward and reverse primers (10 μm) 1 μL each, template DNA 1 μl, sterile water 7 μl. PCR procedure was as follows: pre-denaturation at 94° C. for 5 min; denaturation at 94° C. for 30 sec, annealing at 58° C. for 30 sec, extension at 72° C. for 90 sec, 35 cycles; extension at 72° C. for 5 min. The PCR products were detected by electrophoresis in 1% (w/w) agarose gel for 30 min, and then stained by ethidium bromide. The electrophoretic bands were observed on the UV transmittance instrument. A 1325-bp fragment (SEQ ID NO: 3) was obtained from the transgenic positive plants by PCR, such as T-1, T-2, T-3 and T-4. No PCR products were amplified by the primers from non-transgenic plants, such as T-5 and T-6 (FIG. 1).

### Example 2. Knockout of the Xa7 Gene in Bacterial-Blight Resistance Rice Variety Zhen-Hui 084

### (1) Construction of CRISPR/Cas9 Knockout Vector of the Xa7 Gene

According to the nucleotide sequence of the Xa7 gene (SEQ ID NO: 1), the CRISPR target site design website (http://www.rgenome.net/cas-designer/) was used to design the gene editing target, and one of the target sequences is Target-1: 5′-CGTATGCCCGTTGCAGTTGCAGG-3′ (SEQ ID NO. 4). The primers sequences were synthesized according to the sequence of Target-1: Target-1 F: 5′-GGCACGTATGCCCGTTGCAGTTGC-3′ (SEQ ID NO. 18) and, Target-1 R: 5′-AAACGCAACTGCAACGGGCATACG-3′ (SEQ ID NO. 19). T4 PNK enzyme was used to carry out 5-hydroxyl terminal phosphorylation (NEB, USA). The 50 μl solution contained 10 μl Target-1 F primer (10 μM), 10 μl Target-1 R primer (10 μM), 1 μl T4 ligase, 5 μl Buffer (coming with the products) and 24 μl ddH₂O, and was incubated at 37° C. for 1 h. After that, 2.5 μl (1M) NaCl was add, incubated at 95° C. for 5 sec and naturally annealed for 2-3 h to obtain the Target-1 annealing probe.

The pCAMBIA1300-pYAO-cas9 vector was obtained from Zhili Zhongte Biotechnology company (Wuhan, China) through commercial purchase, and the link is http://www.zlzt.com/plasmid/z1-040598.html. This vector was digested by restriction endonuclease BsaI (NEB, USA) according to the manufacturer's instructions. The linearized vector fragment was recovered from by gel cutting and purified by AxyPrep DNA Gel Recovery Kit (Axygen, USA). The Target-1 probe annealled above and the vector fragment digested be enzyme were ligated with T4-ligase reagents (NEB, USA) and transformed into DH5a competent cells. The positive clones obtained were sent to biotechnology companies for sequencing. If the inserted fragment in the vector was 5′-CGTATGCCCGTTGCAGTTGCAGG-3′ (SEQ ID NO. 4), the construction of the vector was judged to be successful, and the Target-1 knockout vector of Xa7 gene was obtained. Namely, the Target-1 knockout vector of Xa7 gene can be directed to edit the Target-1 site in Xa7 gene (SEQ ID NO: 1) by CRISPR/Cas9 technology, resulting in mutations.

According to the same method above, another gene-editing target sequence was designed in the different site of Xa7 gene as Target-2: 5′-CCAGTTCCCGCGCGCCGCTGGGG-3′ (SEQ ID NO. 5). The primers sequences were synthesized according to the sequence of Target-2: Target-2 F: 5′-GGCACCAGTTCCCGCGCGCCGCTG-3′ (SEQ ID NO. 20) and, Target-2 R: 5′-AAACCAGCGGCGCGCGGGAACTGG-3′ (SEQ ID NO. 21). The Xa7-gene knockout vector for Target-2 was constructed. If the sequencing result shows that the inserted fragment in the vector is 5′-CCAGTTCCCGCGCGCCGCTGGGG-3′ (SEQ ID NO. 5), the construction of the vector is judged to be successful. The Target-2 knockout vector of Xa7 gene can be directed to edit the Target-2 site in Xa7 gene (SEQ ID NO: 1) by CRISPR/Cas9 technology, resulting in mutations.

### (2) Rice Genetic Transformation and Identification of Knockout Plants

The two knockout vectors of the Xa7 gene obtained above were respectively transformed into the bacterial-blight resistance rice variety Zhen-hui 084 by the method reported by Nishimura et al. (Nishimura et al, Nat Protoc, 2006) to get the transgenic rice plants.

Genomic DNAs were extracted from the leaves of transgenic plants according to the method of Panaud et al. (Mol Gen Genet, 1996). The primers were synthesized as follows: forward: 5′-ATGGCGGCCGCTGATCATCC-3′ (SEQ ID NO. 22) and reverse: 5′-TTAATTGCCACCGATGAGGTAATC-3′ (SEQ ID NO. 23). The Xa7 gene sequences (SEQ ID NO: 1) of very transgenic plants were amplified by PCR using the proof-reading Taq enzyme PrimeSTAR® HS DNA Polymerase (TaKaRa, Japan), and the mutations of Target-1 and Target-2 sites in the gene were analyzed. Total 50 μl PCR solution contains PrimeSTAR HS (Premix) 25 μl, forward and reverse primers (10 μm) 1 μl, template DNA 2 μl (<200 ng), sterile water 214 PCR procedure was as follows: pre-denaturation at 95° C. for 5 min; denaturation at 98° C. for 10 sec, annealing at 60° C. for 15 sec, extension at 72° C. for 30 sec, 30 cycles; extension at 72° C. for 5 min.

The PCR products from transgenic plants were connected into T-Vector PMD18 (TaKaRa, Japan), and the ligation solutions and reaction conditions were operated according to the manufacturer's instructions. The ligation products of 10 μl were transformed into JM109 competent cells by the heat-shock method, and the positive clones were sent to biotechnology companies for sequencing.

Serval editing types were found at the knockout sites of the Xa7 gene in transgenic plants, as shown in FIG. 2, all of which led to frameshift mutation or fragment deletion of the Xa7 gene. Finally, four kinds of Xa7-knockout mutants were obtained named as ko-1, ko-2, ko-3 and ko-4.

### Example 3. Bacterial-Blight Resistance Identification of the Two Kinds of Transgenic Rice Plants

The Xa7 transgenic plants obtained above (the transgenic positive plants obtained from Example 1) and the control variety Zhong-hua 11, Xa7-knockout plants (ko-1, ko-2, ko-3, ko-4) and the control variety Zhen-hui 084 were planted in summer field at temperatures of 28° C. to 37° C. At tillering stage of rice, the fully expanded new leaves of the plants were selected to inoculate with Xoo (Xanthomonas campestris pv. Oryzae) strain PXO86 using leaf-cutting method (Kauffman et al., Plant Dis Rep, 1973). The PXO86 strain contains an avirulent factor AvrXa7 which can activate the resistance of Xa7 gene. Two weeks after inoculation, the lesion lengths of inoculated leaves were measured downward from the cut sites of the inoculated leaves, and evaluated according to the standard: resistant (≤3.0 cm), moderately resistant (3.1 to 6.0 cm), moderately susceptible (6.1 to 9.0 cm), and susceptible (>9.0 cm).

The identification results are as follows: the susceptible variety Zhong-hua 11 is highly susceptible to PXO86 (FIG. 3), the transgenic positive plants of Zhong-hua 11 with Xa7 gene are highly resistant to PXO86 (FIG. 3); the resistant variety Zhen-hui 084 is highly resistant to PXO86 (FIG. 4), the mutant plants of Zhen-hui 084 knockout the Xa7 gene are highly susceptible to PXO86 (FIG. 4). The results indicated that rice plants containing the Xa7 gene or being transformed with an exogenous Xa7 gene can obtain highly resistant to bacterial blight. It has a great application value in breeding.

Finally, it is important to note that the above description is only specific embodiments of the present disclosure. Obviously, the disclosure is not limited to the above embodiments, but can also have a lot of deformation. All the deformation that the general technical personnel in this field can directly derive or associate with the contents disclosed in this field should be considered as the scope of protection of the disclosure.

1. A recombinant vector comprising a nucleotide sequence encoding the amino acid sequence of SEQ ID NO: 2.

2. The recombinant vector of claim 1, wherein the recombinant vector comprises the nucleotide sequence of SEQ ID NO: 1.

3. The recombinant vector of claim 2, wherein the recombinant vector comprises the nucleotide sequence of SEQ ID NO: 3.

4. The recombinant vector of claim 1, wherein the recombinant vector is a plasmid.

5. A transgenic rice plant comprising the recombinant vector of claim 1.

6. A method for increasing resistance of a rice plant against the bacterial-blight disease, comprising:
transforming a recombinant vector comprising a nucleotide sequence encoding the amino acid sequence of SEQ ID NO: 2 into the rice plant, resulting a transgenic rice plant capable of expressing a protein having the amino acid sequence of SEQ ID NO: 2.

7. The method of claim 6, further comprising amplifying the nucleotide sequence of SEQ ID NO: 1 through polymerase chain reaction.

8. The method of claim 7, further comprising introducing the amplified nucleotide sequence into a gene expression vector.

9. The method of claim 7, wherein amplification is performed through a pair of primers as set forth in SEQ ID NOs: 14 and 15.

10. The method of claim 6, wherein the recombinant vector is plasmid.

11. The method of claim 6, wherein the recombinant vector comprises the nucleotide sequence of SEQ ID NO: 1.

12. The method of claim 11, wherein the recombinant vector comprises the nucleotide sequence of SEQ ID NO: 3.


SEQUENCE LISTING (23 sequences)

SEQ ID NO: 1 - DNA, 342 bp, Oryza sativa
  source 1..342: mol_type = genomic DNA
atggcggccgctgatcatcctgatcgtatgcccgttgcagttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgctggggtcgccgtggccattgccagggcgctgcaggattacctcatcggtggcaattaa

SEQ ID NO: 2 - protein, 113 aa, Oryza sativa
MAAADHPDRMPVAVAGLRHHYAFPANLRPAARLLTVNSGVFLISTAGAIVLVHTAGNPPAIDNDPAYALVAFVLFLLGIWLMSIALVADQFPRAAGVAVAIARALQDYLIGGN

SEQ ID NO: 3 - DNA, 1325 bp, Oryza sativa
  source 1..1325: mol_type = genomic DNA
gttcggctgcggcctgcagtggctggcaaaaatctcgaactgctctgctcaagtgcctcaactggcagagataaatcctaaaaaaactgaaaaataggccggtatcccagctcttctgctgaaaaaaactgaaaaactgggtgtaagattgatcgcaaaagtacttctggcacttgtcattttcgccacttctttggtctcttcgtcctatttttgacatttctcttcgtccttttttcttttctctttcaaacgtgcagtcgccgtcgaggacggtgaaagccctgactgctaaaaccaatatataacccccccccccccagataaccacatacgaacgaaggctttgaagcatcgcacacttgaagagcccccttcccaaccacagccagcggtttccaaactccacgcctcgctcaacctgggggatccatcatccatggcggccgctgatcatcctgatcgtatgcccgttgcagttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgctggggtcgccgtggccattgccagggcgctgcaggattacctcatcggtggcaattaactagaagcttcgaccatggctctgcacattcctctgctccagttgttcccggcttcccgtacgtgtgcctgatgattgtctttctctgtttatttggctagtattttaggcttggaagttgaaaaactgtaaatctgcttctttttcccctctgtactactactagactttcttttttaagctgacgtcatacacacaccccagattcataacgtgtcgtatagtaaatgtattcgaggcttgtaataaaaaggcacccgtaggtgtatgctctgttcagtctgatgttctaaataatcaagatactattagtcgtattttctgcttcatggcggcagtggtggtaatatttcaattccatacgaagggtcctcgctgactcactgtgtggtgtgcgtgctcatttgctgacatgctaaagtcggactatagccaggtgtggccgtgagggcattcggtgtactgaaaatagtgcaaaaacatccgccgcggtcgcgccaagtttgtcgagaatacgcacaccgaaccggcctcacaactgatt

SEQ ID NO: 4 - DNA, 23 bp, Oryza sativa
  misc_feature 1..23: note = Target-1
  source 1..23: mol_type = genomic DNA
cgtatgcccgttgcagttgcagg

SEQ ID NO: 5 - DNA, 23 bp, Oryza sativa
  misc_feature 1..23: note = Target-2
  source 1..23: mol_type = genomic DNA
ccagttcccgcgcgccgctgggg

SEQ ID NO: 6 - DNA, 343 bp, synthetic construct
  misc_feature 1..343: note = ko-1 #1
  source 1..343: mol_type = other DNA
atggcggccgctgatcatcctgatcgtatgcccgttgcagtctgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgctggggtcgccgtggccattgccagggcgctgcaggattacctcatcggtggcaattaa

SEQ ID NO: 7 - DNA, 340 bp, synthetic construct
  misc_feature 1..340: note = ko-1 #2
  source 1..340: mol_type = other DNA
atggcggccgctgatcatcctgatcgtatgcccgttgcaggcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgctggggtcgccgtggccattgccagggcgctgcaggattacctcatcggtggcaattaa

SEQ ID NO: 8 - DNA, 343 bp, synthetic construct
  misc_feature 1..343: note = ko-2 #1
  source 1..343: mol_type = other DNA
atggcggccgctgatcatcctgatcgtatgcccgttgcagtttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgctggggtcgccgtggccattgccagggcgctgcaggattacctcatcggtggcaattaa

SEQ ID NO: 9 - DNA, 328 bp, synthetic construct
  misc_feature 1..328: note = ko-2 #2
  source 1..328: mol_type = other DNA
atggcggccgctgatcatcctgatcgtatgcccgttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgctggggtcgccgtggccattgccagggcgctgcaggattacctcatcggtggcaattaa

SEQ ID NO: 10 - DNA, 343 bp, synthetic construct
  misc_feature 1..343: note = ko-3 #1
  source 1..343: mol_type = other DNA
atggcggccgctgatcatcctgatcgtatgcccgttgcagttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgcctggggtcgccgtggccattgccagggcgctgcaggattacctcatcggtggcaattaa

SEQ ID NO: 11 - DNA, 312 bp, synthetic construct
  misc_feature 1..312: note = ko-3 #2
  source 1..312: mol_type = other DNA
atggcggccgctgatcatcctgatcgtatgcccgttgcagttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgtggccattgccagggcgctgcaggattacctcatcggtggcaattaa

SEQ ID NO: 12 - DNA, 338 bp, synthetic construct
  misc_feature 1..338: note = ko-4 #1
  source 1..338: mol_type = other DNA
atggcggccgctgatcatcctgatcgtatgcccgttgcagttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgggtcgccgtggccattgccagggcgctgcaggattacctcatcggtggcaattaa

SEQ ID NO: 13 - DNA, 343 bp, synthetic construct
  misc_feature 1..343: note = ko-4 #2
  source 1..343: mol_type = other DNA
atggcggccgctgatcatcctgatcgtatgcccgttgcagttgcaggcttgcgccaccattacgccttccctgcaaaccttcgccccgccgctcgactgctgaccgtcaactccggcgtcttcctcatctccaccgccggggccatcgtcctcgtccacaccgccggtaacccacccgccatcgacaacgatccagcctacgccttggtcgcattcgtgctcttcctcctcggaatctggctcatgtctattgccctcgtcgccgaccagttcccgcgcgccgtctggggtcgccgtggccattgccagggcgctgcaggattacctcatcggtggcaattaa

SEQ ID NO: 14 - DNA, 36 bp, synthetic construct
  misc_feature 1..36: note = primer
  source 1..36: mol_type = other DNA
cggccagtgccaagcttgttcggctgcggcctgcag

SEQ ID NO: 15 - DNA, 37 bp, synthetic construct
  misc_feature 1..37: note = primer
  source 1..37: mol_type = other DNA
tgaccacccggggatccaatcagttgtgaggccggtt

SEQ ID NO: 16 - DNA, 19 bp, synthetic construct
  misc_feature 1..19: note = primer
  source 1..19: mol_type = other DNA
gttcggctgcggcctgcag

SEQ ID NO: 17 - DNA, 20 bp, synthetic construct
  misc_feature 1..20: note = primer
  source 1..20: mol_type = other DNA
aatcagttgtgaggccggtt

SEQ ID NO: 18 - DNA, 24 bp, synthetic construct
  misc_feature 1..24: note = primer
  source 1..24: mol_type = other DNA
ggcacgtatgcccgttgcagttgc

SEQ ID NO: 19 - DNA, 24 bp, synthetic construct
  misc_feature 1..24: note = primer
  source 1..24: mol_type = other DNA
aaacgcaactgcaacgggcatacg

SEQ ID NO: 20 - DNA, 24 bp, synthetic construct
  misc_feature 1..24: note = primer
  source 1..24: mol_type = other DNA
ggcaccagttcccgcgcgccgctg

SEQ ID NO: 21 - DNA, 24 bp, synthetic construct
  misc_feature 1..24: note = primer
  source 1..24: mol_type = other DNA
aaaccagcggcgcgcgggaactgg

SEQ ID NO: 22 - DNA, 20 bp, synthetic construct
  misc_feature 1..20: note = primer
  source 1..20: mol_type = other DNA
atggcggccgctgatcatcc

SEQ ID NO: 23 - DNA, 24 bp, synthetic construct
  misc_feature 1..24: note = primer
  source 1..24: mol_type = other DNA
ttaattgccaccgatgaggtaatc